(19) United States
(12) Patent Application Publication
(10) Pub. No.: US 2002/0037519 A1
(43) Pub. Date: Mar. 28, 2002

(54) Identifying clusters of transcription factor binding sites

(76) Inventor: David J. States, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/853,141

(22) Filed: May 10, 2001

Related U.S. Application Data
(60) Provisional application No. 60/203,469, filed on May 11, 2000.

(51) Int. Cl. C12Q 1/68; G06F 19/00; G01N 33/48; G01N 33/50

(52) U.S. Cl. 435/6; 702/20

(57) ABSTRACT

Identifying clusters of protein binding sites in a nucleotide sequence under analysis. A computerized system determines likelihood parameters for a plurality of known protein binding sites. The likelihood parameter for each protein binding site represents a likelihood that the protein binding site will occur in a nucleotide sequence under analysis relative to a likelihood that the protein binding site will occur in a random nucleotide sequence of a substantially equivalent composition. Selected protein binding sites are grouped as a function of their respective likelihood parameters to determine a likelihood score, which is compared to a predetermined threshold. The selected protein binding sites in the nucleotide sequence are identified as one or more clusters if the likelihood score exceeds the predetermined threshold.

### CROSS-REFERENCE TO RELATED APPLICATION

This application claims the benefit of commonly assigned Provisional Patent Application Ser. No. 60/203,469, filed May 11, 2000, the entire disclosure of which is incorporated herein by reference.

### STATEMENT REGARDING FEDERALLY SPONSORED RESEARCH OR DEVELOPMENT

This invention was made in part with Government support under grants R01-HG01391 and DE-FG02-94ER61910, awarded by the National Institutes of Health and the Department of Energy, respectively. The Government has certain rights in this invention.

### BACKGROUND OF THE INVENTION

Gene expression is regulated by the cooperative action of sequence specific DNA-binding of proteins to genomic DNA. While individual DNA-binding proteins may exhibit binding that is sequence specific, eukaryotic gene regulation appears, in most cases, to be regulated by complexes of DNA-binding proteins rather than by the sequence specific binding of individual proteins. The ability to recognize and characterize clusters of protein binding sites in the genome is, therefore, an important step in the functional analysis of genomic sequence data. However, the limited selectivity of individual DNA-binding proteins makes it difficult to recognize and analyze regulatory sites in complex genomes.

Proteins that bind to DNA and act as both enhancers and repressors of gene transcription have been identified in bacterial and eukaryotic systems. The regulation of expression for many bacterial genes is often mediated by binding of a single protein to a single site. Even in bacterial systems, cooperative binding of proteins to adjacent sites plays an important role in enhancing the specificity of protein binding. Eukaryotic genomes are larger and more complex than bacterial genomes, and the expression of eukaryotic genes is typically regulated by the binding of complexes of several DNA-binding proteins acting cooperatively.

Moreover, protein binding sites in the eukaryotic genome may alter the expression level of genes located tens of kilobases away on the genome. This further complicates the identification of significant binding site clusters.

Cooperative binding allows combinatorial control and may facilitate adaptive evolution. In addition, cooperative binding increases the specificity of binding by requiring multiple sites to occur in adjacent locations. Several factors are believed to contribute to the evolution of regulatory mechanism utilizing cooperative binding in eukaryotic organisms with large genome sizes. For example, physical size may be a factor. To define a binding site that is expected to be unique in a sequence the size of the human genome, the site must be at least 15 nucleotides long. However, a DNA double helix of this length is physically larger than most globular proteins, which prevents sequence specific contacts from being defined over such an extended region. The energetics of macromolecular interactions may also be a factor in the regulatory mechanism using cooperative binding. Sequence specific binding of proteins to DNA depends on the relative binding energies for specific and non-specific binding modes. For a protein to bind to a unique site specifically in the human genome, the affinity for that site must exceed the affinity for non-specific binding sufficiently so that the specific site is occupied. Inasmuch as the haploid human genome is approximately three billion basepairs in size, the site-specific binding energy would need to exceed the non-specific binding energy by about 13 kcal/M. This site-specific binding energy is greater than the typical folding energy for most globular proteins. Cooperative binding may also facilitate the recruitment of different molecular elements and functions to a complex (DNA-binding, activation domains, scaffold protein association, etc.).

Many DNA-binding proteins have been identified in eukaryotic organisms. For example, the TRANSFAC transcription factor database, maintained at the GBF Braunschweig, Germany, defines sequence specific binding site patterns for more than 200 such proteins. The TRANSFAC database, available via the Internet at http://transfac.gbf.de/, includes transcription factors, their genomic binding sites, and DNA-binding profiles. Typically, the binding profiles defined for individual transcription factors are relatively short and allow for multiple residues at many positions. In a search of random DNA sequence, matches comparable to biologically functional sites are expected to occur with high frequency. In order to analyze transcriptional regulation of the human genome, methods are needed for identifying and defining statistically significant clusters of protein-binding sites in the human genome.

The density of transcription factor binding sites near promoters have been found to be higher than the genome as a whole. As such, incorporating searches for transcription factor binding site clusters into algorithms for promoter recognition can improve the performance in correctly recognizing functional transcription start sites. Also, oligomer frequency models for promoter recognition may implicitly capture information on the prevalence of transcription factor binding sites.

Models based on the cooperative binding of multiple transcription factors have been developed to search for specific regulatory sites in genomic sequence. For example, Wagner, “A computational genomics approach to the identification of gene networks,” Nucleic Acids Res. 25(18), 3594-604 (1997), describes a statistical approach to searching for transcription factor binding site clusters in yeast. Also, the COMPEL database, maintained at the Institute of Cytology and Genetics, RAS, Novosibirsk, Russia, contains composite regulatory elements (i.e., pairs of closely situated sites and transcription factors binding to them).

Moreover, transcription factor binding sites clusters represent a novel class of candidate loci for disease genes. Transcription factor binding site clusters are likely to play important regulatory roles and several genes associated with quantitative trait loci have been identified in non-coding regulatory regions of the genome (TNF-alpha, LTC4S). Mutations mapping to enhancers have been identified in the thalassemias and muscular dystrophy. Further, enhancer rearrangements activating the expression of oncogenes and leading to leukemia have been discussed.

In view of the foregoing, further improvements in searching for transcription factor binding sites are needed, including a computational efficient system that identifies clusters of binding sites in extended regions of sequence by deriving statistics for assessing the significance of clusters of protein-binding sites.

### SUMMARY OF THE INVENTION

The invention meets the above needs and overcomes the deficiencies of the prior art by providing improved identification of transcription factor binding site clusters, particularly in the human genome. According to one aspect of the invention, statistically significant clusters of protein binding sites are identified from genomic data. The ability to recognize and characterize clusters of protein binding sites in the genome is an important step in the functional analysis of genomic sequence data. Moreover, the collection and reporting of identified clusters will provide great benefits to future research. Advantageously, the present invention utilizes known databases of sequence and transcription factor data as well as a computationally efficient search algorithm. Moreover, identification of transcription factor binding site clusters as described herein is economically feasible and commercially practical.

Briefly described a method embodying aspects of the invention identifies clusters of protein binding sites in a nucleotide sequence under analysis. The method includes determining likelihood parameters for a plurality of known protein binding sites. The likelihood parameter for each protein binding site represents a likelihood that the protein binding site will occur in the nucleotide sequence under analysis relative to a likelihood that the protein binding site will occur in a random nucleotide sequence of a substantially equivalent composition. The method also includes grouping selected protein binding sites as a function of their respective likelihood parameters to determine a likelihood score and comparing the likelihood score to a predetermined threshold. If the likelihood score exceeds the predetermined threshold, then the selected protein binding sites are identified as one or more clusters.

In another form, the invention is directed to a computer readable medium having computer-executable instructions for performing the method of identifying clusters.

In yet another form, a method of identifying protein binding sites in a nucleotide sequence under analysis includes determining likelihood parameters for a plurality of known protein binding sites and comparing the likelihood parameters to a predetermined threshold to select the protein binding sites that have a substantially greater relative likelihood of occurrence. The method also includes defining an index that references segments of the nucleotide sequence, searching the index to find the segments that are similar to one or more of the selected protein binding sites, and identifying the segments found in the index search as protein binding sites in the nucleotide sequence based on the index search.

A data structure embodying aspects of the invention includes individual protein binding sites information and cluster information. The individual protein binding sites are identified in a nucleotide sequence under analysis where each protein binding site has a sequence that corresponds to a portion of the nucleotide sequence under analysis. The cluster information identifies clusters of the protein binding sites in the nucleotide sequence under analysis. The clusters are identified from the protein binding sites as a function of likelihood parameters for the protein binding sites.

Alternatively, the invention may comprise various other methods and apparatuses.

Other objects and features will be in part apparent and in part pointed out hereinafter.

### BRIEF DESCRIPTION OF THE DRAWINGS

FIG. 1 is a block diagram of a computer system embodying aspects of a preferred embodiment of the invention. FIGS. 2A and 2B illustrate an exemplary flow diagram of the operation of the computer system of FIG. 1.

FIG. 3 illustrates an exemplary analysis of the human MHC class 1 locus (GenBank accession AF055066), including the locations of annotated genes, protein binding site clusters, and CpG islands, developed by the system of FIG. 1.

FIG. 4 illustrates an exemplary analysis of the G6PD region on human Xq28 (GenBank accession HUMFLNG6PD), including the locations of annotated genes, protein binding site clusters, and CpG islands, developed by the system of FIG. 1.

FIG. 5 illustrates an exemplary analysis of the human SCL gene, including the locations of coding exons, protein binding site clusters, and CpG islands, developed by the system of FIG. 1.

FIG. 6 illustrates an exemplary analysis of the mouse SCL gene, including the locations of coding exons, protein binding site clusters, and CpG islands, developed by the system of FIG. 1.

FIG. 7 is a two-dimensional comparison of the human and mouse SCL gene structures of FIGS. 5 and 6, respectively, for a one kilobase window around the transcription start site.

Corresponding reference characters indicate corresponding parts throughout the drawings.

### DETAILED DESCRIPTION OF THE INVENTION

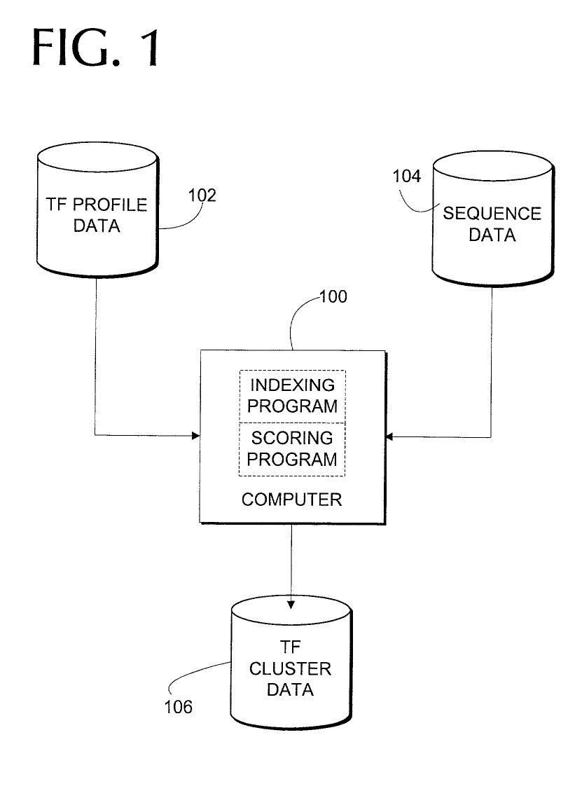

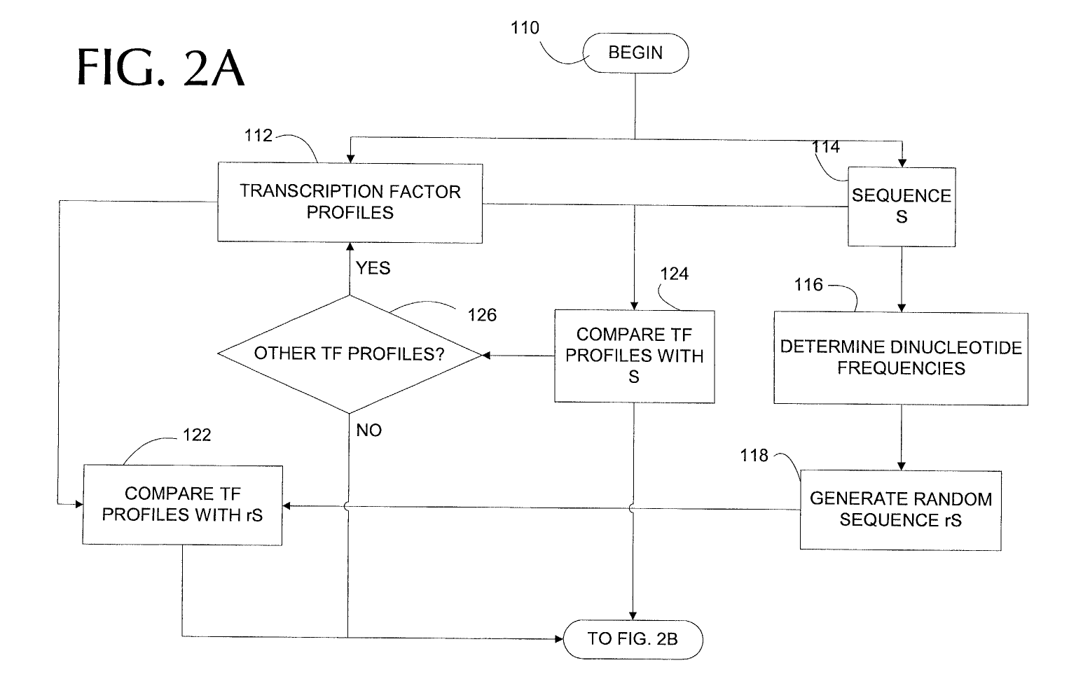

Referring now to the drawings, FIG. 1 illustrates an exemplary physical configuration of a computer system embodying aspects of the invention. A conventional data communication network couples a computer 100 to a first database 102 and a second database 104. In this exemplary environment, the network is a global communication network such as the Internet. The computer 100 preferably has one or more processors and at least some form of computer readable media. For example, computer 100 has a system memory including read only memory (ROM) and random access memory (RAM). The computer 100 may also include other removable/non-removable, volatile/nonvolatile computer storage media such as a hard disk drive that reads from or writes to non-removable, nonvolatile magnetic media and a magnetic and/or optical disk drive that reads from or writes to a removable, nonvolatile disk. Networking environments are commonplace in offices, enterprise-wide computer networks, intranets, and global computer networks (e.g., the Internet). A conventional physical data link or connection, such as a modem and telephone line, T-1, ISDN, or the like, may be used for connecting computer 100 to databases 102, 104. Generally, the data processors of computer 100 are programmed by means of instructions stored at different times in the various computer-readable storage media of the computer.

The database 102 preferably contains transcription factor profile data. As described above, many DNA-binding proteins have been identified in eukaryotic organisms. For example, the TRANSFAC transcription factor database, maintained at the GBF Braunschweig, Germany, defines sequence specific binding site patterns, or motifs, for more than 200 such proteins. The TRANSFAC database, available via the Internet at http://transfac.gbf.de/, includes transcription factors, their genomic binding sites, and DNA-binding profiles. The transcription factor binding site profiles are typically about six to fifteen nucleotides in length. In a preferred embodiment of the invention, only vertebrate sites with relative entropy of 8 bits, for example, are considered. In the TRANSFAC database, 171 vertebrate DNA-binding sites have a relative entropy threshold of at least 8 bits, which represents sufficient information content in the binding site profile.

On the other hand, database 104 preferably contains sequence data from which the sequence under analysis is obtained. Those skilled in the art are familiar with GENBANK, which is the National Institute of Health genetic sequence database available on the World Wide Web for searching at http://www.ncbi.nlm.nih.gov/Genbank/GenbankSearch.html. In this instance, the sequence is approximately 750 kilobases. Although many aspects of the present invention are described in connection with the human genome, it is to be understood that the invention may be used for identifying clusters of transcription factor binding sites in genomes of other eukaryotic organisms.

According to the invention, computer 100 executes computer-readable instructions to identify clusters of protein binding sites in a genomic sequence. In general, the present invention incorporates a detailed two process model to describe the spacing between binding sites to recognize transcription factor binding site clusters in the sequence under analysis. The invention is based on an explicit likelihood model and, thus, permits analytically predicting the frequency with which sites having a particular likelihood score would occur in searching random sequence. Setting the likelihood score threshold to a value at which matches are quite unlikely to have arisen as random fluctuations, several thousand clusters may be identified in the human genome, for example. In this manner, the invention identifies clusters of protein-binding sites in a nucleotide sequence under analysis and derives statistics for assessing their significance.

Likelihood parameters are first determined for a plurality of known protein binding sites from the database 102 and then compared to a predetermined threshold. The likelihood parameter for each protein binding site represents a likelihood that the protein binding site will occur in the nucleotide sequence under analysis, obtained from the database 104, relative to a likelihood that the protein binding site will occur in a random nucleotide sequence of a substantially equivalent composition. By comparing the likelihood parameter to the predetermined threshold, the present invention is able to select the protein binding sites that have a substantially greater relative likelihood of occurrence. According to one embodiment of the invention, an index that references segments of the nucleotide sequence is then searched to find the segments that are similar to one or more of the selected protein binding sites for identifying protein binding sites. This embodiment of the invention further defines sets of selected protein binding sites and optimizes a cumulative likelihood parameter for each set to identify clusters. A database 106 preferably stores the identified protein binding site clusters associated with the sequence under analysis. Although the transcription factor clusters database 106 is shown separately from computer 100, in other embodiments of the invention, database 106 is contained within computer 100.

Referring now to FIGS. 2A and 2B, an exemplary flow diagram illustrates the operation of computer 100 to identify clusters of protein binding sites in a nucleotide sequence under analysis. Beginning at 110, computer 100 proceeds to 112 to obtain transcription factor profiles from database 102 (e.g., TRANSFAC) and proceeds to 114 to obtain a sequence from database 104 (e.g., GENBANK) for analysis. As described in detail below, the present invention identifies protein binding sites in a sequence under analysis using a log likelihood scoring system to evaluate matches of the known binding profiles to sequence data. The score for matching a segment of sequence data is the log of the likelihood ratio that the sequence is derived from a model describing a protein's binding profile relative to the likelihood that it is derived from a null model for the genome.

The computer 100 executes 116 to determine the dinucleotide frequencies for the sequence and executes 118 to generate a random sequence. In this instance, the null model is a first order Markov model with the dinucleotide frequencies determined for each individual sequence. Specific information on determining a likelihood parameter for the plurality of known protein binding sites is provided below. The likelihood parameter for each protein binding site represents a likelihood that the protein binding site will occur in the nucleotide sequence under analysis relative to a likelihood that the protein binding site will occur in a random nucleotide sequence of a substantially equivalent composition. Steps 116 and 118 correspond generally to equation [1] below. In equation [1], a log likelihood L(S) represents the likelihood score for matching the nucleotide sequence S with the protein binding site sequence relative to the null model.

Scores for statistically defined genome features are preferably reported in bits. A bit is a unit of information defined as the information derived from knowing that an event will occur with a likelihood of ½. A feature with 10 bits of information is likely to occur at random one in 1,024 trials (2¹⁰), and a feature with 32 bits of information is unlikely occur in a random sequence collection the size of the human genome. To report the score in bits, the logs are preferably calculated base 2. Also, the null model frequencies are preferably calculated independently for each sequence being analyzed because genomic sequences exhibit patchiness in composition and the dinucleotide frequencies will vary for the different sequences under analysis. At 122, computer 100 performs a direct comparison of the known transcription factor profiles to the random sequence.

Referring now to 124, computer 100 also performs a direct comparison of the known binding profiles to the sequence under analysis. Preferably, computer 100 cycles through each of the profiles, numbering 171 in the TRANSFAC database, as indicated at 126.

Based on the comparisons, computer 100 determines a likelihood score for each match at 130. Step 130 generally corresponds with equation [3] below for determining the combined likelihood score Lₛₑₜ. Following each match, computer 100 updates Lₛₑₜ at 132 and then applies various rules for defining clusters at 134. The profiles are grouped into clusters using a likelhood model that views the cluster as a chain of events that can either be extended or terminated after each binding site. For example, the dynamic programming algorithm used here to assemble sets of close, but not overlapping, binding sites determines the optimal scoring set. Often, there are alternative sets with a nearly optimal score. Transcription factor expression is often highly tissue specific. Binding sites that are not in the optimal set may, nevertheless, be biologically functional features with the precise set of binding sites governing expression in a tissue being determined by transcription factors expressed in that tissue. For many well known transcription factors, the binding site does not meet the threshold of 8 bits of relative entropy employed here. These factors may contribute additional functionality to the binding site clusters defined here.

The analysis presented here is generally conservative in several ways. At 134, for example, protein binding sites are not allowed to overlap at all, but physical binding of transcription factors to opposite faces of the DNA may allow overlapping binding to occur. Many of the transcription motifs in TRANSFAC exhibit dyad symmetry reflecting a dimeric structure of the bound proteins. Although the occurrence of matches on opposite strands of the DNA are not independent for these sites, they are treated as such, which increases the expected frequency of random binding site matches. While database 102 has expanded in recent years to include several hundred motifs, this is still a small fraction of the several thousand DNA binding proteins expected to be present in the human genome. As the number of transcription factor binding sites patterns in database 102 increases, the power of binding site cluster analysis is expected to improve.

Proceeding to 138, the likelihood parameter determined previously at 130 is compared to a predetermined threshold (e.g., 20 bits). In a preferred embodiment of the invention, a computationally efficient search is derived using a neighborhood table to store all possible sequence segments that could be part of a high scoring match to a protein binding site profile. For example, the search routine employed by computer 100 is similar to the well known Basic Local Alignment Search Tool (BLAST) set of search programs for detecting sequence relationships in the available sequence databases. By comparing the likelihood parameter to the predetermined threshold, the present invention is able to select the protein binding sites that have a substantially greater relative likelihood of occurrence. In this manner, the present invention identifies clusters of protein binding sites and reports them at 140. Preferably, the identified clusters are stored in the database 106.

Referring further to the determination of a likelihood parameter, the log likelihood L(S) is defined according to the following:

$$L ( S ) = {\sum }_{i = 1 , l} log ( {p}_{i} ( {s}_{i} ) ) - log ( f ( {s}_{1} ) ) {\sum }_{i = 2 , l} log ( f ( {s}_{i} \vert {s}_{i - 1} ) ) \qquad [ 1 ]$$

where L(S) is the log likelihood score for matching the nucleotide sequence S composed of residues sᵢ to sₘ with the protein binding site sequence of length 1; sᵢ is a residue at position i in the nucleotide sequence; pᵢ(s) is the probability of the residue s at position i in the protein binding site sequence; f(s) is the frequency of the residue s in the nucleotide sequence as a whole; and f (s|s′) is the conditional probability for finding the residue s in the nucleotide sequence as a whole given previous residues s′. To report the score in bits, the logs are calculated base 2. Also, the null model frequencies are preferably calculated independently for each sequence being analyzed because genomic sequences exhibit patchiness in composition and the dinucleotide frequencies will vary for the different sequences under analysis.

For example, the probabilities of the null model are calculated from the occurrence data in the database of binding sites (e.g., TRANSFAC Release 4.0 matrix.dat file). This database lists the number of binding site occurrences for each nucleotide at each position in the motif, or binding site pattern. Occurrence data are converted to a log odds matrix using a pseudocount of 0.5:

$${p}_{i} ( r ) = \frac{{n}_{i} ( r ) + 0.5}{{N}_{i} + 2} \qquad [ 2 ]$$

where nᵢ(r) is the number of occurrences of residue r at site i in the profile data and Nᵢ is the total number of residues of all identities for site i. Compared to the standard Laplace prior using a pseudocount of 1.0, the use of pseudocount of 0.5 results in a bias toward a single dominant residue in the profile probabilities.

As described above, only vertebrate sites with relative entropy of 8 bits are considered. There are, for example, 171 vertebrate DNA-binding sites that meet the 8-bit relative entropy threshold in the TRANSFAC database.

To assess the likelihood of a set of protein binding sites, a combined log likelihood score is calculated. Each individual binding site has a log likelihood score that the sequence of the site was drawn from the distribution described the binding motif relative to the model for random sequence. The log likelihood Lₛₑₜ for observing a set of n sites is

$${L}_{set} = {L}_{1} - log ( 2 m ) + ( {\sum }_{i = 2 , n} {L}_{i} - log ( 2 m ) + log ( 1 - {p}_{term} ) - log ( g ( {x}_{i} - {x}_{i - 1} ) ) ) + log ( {p}_{term} ) \qquad [ 3 ]$$

where Lᵢ is the log likelihood score for site i; xᵢ is the position of site i; m is the number of sites being searched (e.g., 171); and n is the number of sites in this set. The −log(2m) terms reflect the fact that we would accept a hit from any of the m motifs in the query set and that both strands of the DNA are searched. The log (1−pₜₑᵣₘ) term is the log likelihood for not terminating a chain of hits, and the log (pₜₑᵣₘ) is the log likelihood for not extending the chain further. The −log(g(xᵢ−xₗ₋₁)) reflects the probability that a pair of sites will be separated by distance xᵢ-xᵢ₋₁.

An examination of protein binding sites in the human genome with high individual scores (i.e., greater than 20 bits, or a probability of occurring less than once in one million nucleotides of random sequence) revealed a strong tendency for high scoring protein binding sites to be near each other in the human genome. Approximately 25% of high scoring sites lie within 200 nucleotides of a second high scoring site even though fewer than 1% of such sites would be expected to lie within 200 nucleotides of each other if the sites were distributed as a random Poisson distribution. An empirically derived mixture of two exponentials is used to describe the waiting time, or spacing, between sites:

$$g ( t ) = \frac{{α}_{1}}{{l}_{1}} exp ( - \frac{t}{{l}_{1}} ) + \frac{{α}_{2}}{{l}_{2}} exp ( - \frac{t}{{l}_{2}} ) \qquad [ 4 ]$$

where α₁ and α₂ are the weights for the two components of the mixture, and l₁ and l₂ are the mean spacings between adjacent sites for the two components of the mixture. These parameters are adjusted empirically to fit the observed spacing between adjacent pairs of sites found in searching the human genome, for example. This suggests that clustering of protein-binding sites occurs at multiple resolutions. Empirical data indicates that approximately 30% of high scoring sites have a nearly adjacent high scoring site with a mean distance between sites of 70 nucleotides while the remaining pairs are distributed with a mean distance of about 4500 nucleotides (i.e., a distance at which sites may be separated by several intervening nucleosomes). The fit is a sum of two exponentials with the functional form f(t)=0.30/70*exp(−t/70)+0.70/4500*exp(−t/4500). Based on these results, a two component mixture model for spacing between sites is used to describe clusters of all protein binding sites. The parameters are adjusted empirically to better represent the higher frequency of sites when low scoring as well as high scoring binding sites are considered. In this instance, the fraction of site spacings described by the short period decay depends on the total number of sites in the pattern set. Values for α₁ and α₂ of 0.9 and 0.1, respectively, with l₁ and l₂ equal to 10 and 300, respectively, are used for subsequent analysis.

The expected number of protein binding site occurrences E of a set with score greater than or equal to Sₛₑₜ in searching L residues of sequence is:

E=Lexp(−Sₛₑₜ). [5]

A dynamic programming algorithm is used to select optimal sets of sites (i.e., those sites having the highest possible score for a particular region, such as 150 kilobases). In particular, sites in a set are not allowed to overlap each other. This is important because TransFac includes a number of redundant sites, and it would not be appropriate to count multiple hits to essentially the same sequence.

Additional protein-binding sites that are not part of the high scoring set are frequently present. These may be overlapping sites, redundant definitions, or sites that were simply not part of the high scoring set. As a second measure of significance, an x² statistic is calculated comparing the number of observed and expected binding sites matches in the sequence interval covered by the high scoring set. The number of expected motif matches Eₘ is determined from the standard distribution:

$${E}_{m} = {l}_{i} erf ( \frac{{S}_{cut} - {\overline{S}}_{m}}{{V}_{m}} ) \qquad [ 6 ]$$

where lᵢ is the length of the interval; Sₘ is the mean score for motif m on random sequence; and Vₘ is the standard deviation of the score for the motif applied to random sequence. This formula gives the number of matches scoring above the single sites score cutoff S_{cut} for the motif in an interval of random sequence with the same size as the high scoring set. Given Nₘ, matches for the motif in the interval, the chi square statistic x² was defined as

$${χ}^{2} = \frac{( {N}_{m} - {E}_{m} )}{{E}_{m}} . \qquad [ 7 ]$$

Additional matching sites are listed in descending order of the statistic. The number of additional protein binding sites seen in a high scoring region often provide additional support for the non-random nature of the binding site cluster.

A computationally efficient search may be implemented using an indexing algorithm. For each weight matrix in the library, the run of 10 consecutive columns, or 10 character words, with the highest relative entropy is identified. For example, the TPANSFAC library contains 171 protein binding profiles. The highest score Lᵣ from the remainder of the matrix is then determined. To search for all matches scoring at least C, all 10 character words matching the most informative segment with a score above C−Lᵣ are stored in an index. If the length of the pattern is less than 10 nucleotides, the pattern is extended to 10 nucleotides by including all suffix strings with zero score. To search a query sequence, incremental segments of 10 characters are used to generate a search word, and this word is used to look up potential hits in the index. The full pattern score for each candidate hit is then evaluated explicitly. Note that this algorithm finds all pattern hits scoring above C. If there are no entries in the index for the search word generated at a particular location in the query sequence, then no pattern in the library could achieve a score of C when tested on the corresponding sequence segment.

Binding site clusters are modeled as chains of single sites with the probability for chain extension and termination weighted to guarantee that the sum over all possible chains is unity. The spacing of individual high scoring sites in genomic sequence suggests that clustering occurs at multiple resolutions, and this is incorporated empirically into a waiting time distribution between sites in clusters. Computer 100 preferably executes a dynamic program at 134 of FIG. 2B to identify optimal sets of binding sites in clusters containing multiple overlapping protein binding sites. A cluster of protein binding sites is regarded as significant if it is unlikely that a similarly scoring cluster would be found searching a random sequence database of similar size. For example, an analysis of the data reveals that 12,574 clusters of protein-binding sites with scores above 20 bits (1 per million chance of occurrence at random) are found in the available human genome sequence (1.8 billion basepairs). of these sites, 5,384 have scores (>32 bits) higher than would be expected in a search of random sequence the size of the human genome. The locations of these clusters correlate with transcription start sites and experimentally defined DnasI hypersensitive sites. In the range of 16 to 20 bits, 7,159 clusters are identified. These clusters fall into a statistical gray zone and may be functional elements of the genome. Many of these clusters are found in close proximity to annotated transcription start sites, suggesting that they are indeed functional.

The accepted estimate for the number of protein binding site clusters in the human genome (40,000-60,000) is roughly comparable to the accepted estimate for the number of genes in the genome (30,000-100,000). There does not appear to be a one to one correspondence between binding site clusters and genes. Instead, some genes are associated with several binding site clusters while others do not appear to be associated with any. Because enhancers may act at considerable distance (e.g., 40 kilobases is the case of the beta globin LCR), it is difficult to define precisely which genes may be controlled by which clusters.

An examination of two well-studied loci, G6PD and MHC (see EXAMPLE 1), demonstrates a strong correlation between the presence of a transcription factor binding site cluster and an annotated transcription start site. While both the G6PD and MHC loci are relatively gene dense, several transcription factor binding site clusters were identified in close proximity to transcription start sites. Further, comparison of the human and mouse SCL genes (see EXAMPLE 2) demonstrates that at least some of the protein binding site clusters identified here are an evolutionarily conserved feature of the genome.

The transcription factor binding site clusters identified in the SCL gene correspond to functionally characterized enhancer elements active in the regulation of this gene. Transcription factor cluster analysis complements comparative sequence analysis. Extensive regions of conserved sequence are seen in the SCL locus although no function has been ascribed to many of these regions. Conversely, some of the elements regulating expression of this gene are not apparent as binding site clusters but they are revealed by comparative sequence analysis.

Transcription factor binding sites clusters represent a novel class of candidate loci for disease genes. Transcription factor binding site clusters are likely to play important regulatory roles, and several genes associated with quantitative trait loci have been identified in non-coding regulatory regions of the genome (TNF-alpha, LTC4S). Mutations mapping to enhancers have been identified in the thalassemias and muscular dystrophy. Further, enhancer rearrangements activating the expression of oncogenes and leading to leukemia have been discussed. In searching for candidate mutations, transcription factor binding site clusters will need to be tested independently because they will not be represented in expresses sequence collections.

As described above, the invention preferably employs a computationally efficient search for identifying all possible sequence segments that could be part of a high scoring match to a protein binding site profile. In a pre-computing stage, computer 100 collects data for a table listing the 171 transcription factor binding pattern weight matrices found in TRANSFAC that have a relative entropy of greater than 8 bits. Computer 100 also lists the number of 10 character words needed to cover all possible hits above 12 bits for this weight matrix in the table as part of an indexing routine. For example, 7.6 million words are generated in an index. Since the size of the index is 4¹⁰ (approximately 1.05 million), the index points to an average 7.6 patterns at each location that will need to be evaluated explicitly if a match is found with the most significant information in the matrix. In contrast, all 171 motifs would need to be evaluated when using a full pattern search. As a result, indexing provides substantial increases in performance and allows, for example, the full HTG division of GENBANK to be searched with all profiles in a few hours using a conventional desktop personal computer (e.g., 500 MHz Intel PENTIUM® II processor).

The following examples are simply intended to further illustrate and explain the present invention. This invention, therefore, should not be limited to any of the details in these examples.

### EXAMPLE 1

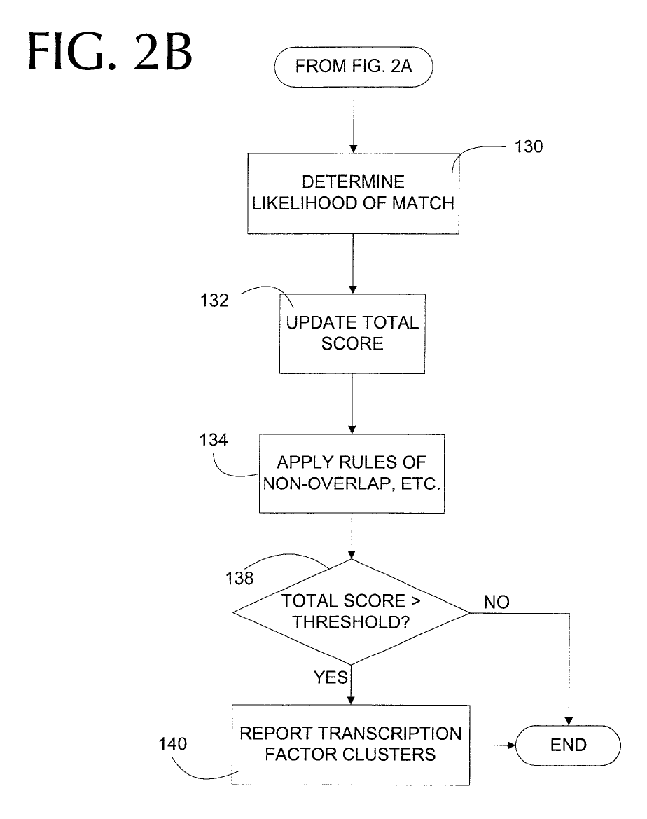

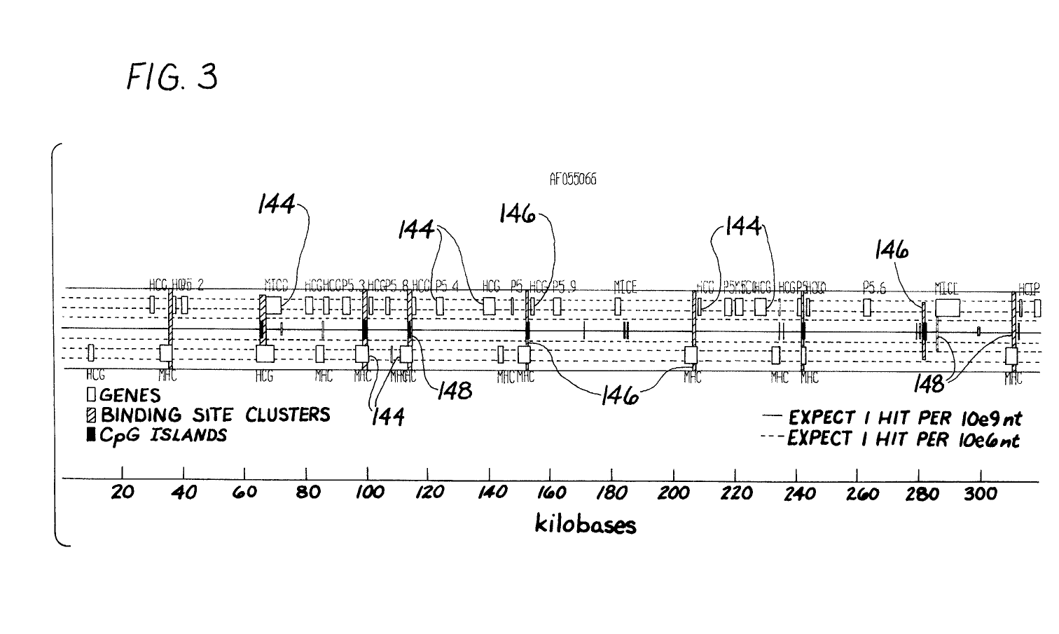

FIG. 3 illustrates an analysis of the human MHC class 1 locus (GenBank accession AF055066), including the locations of annotated genes, protein binding site clusters, and CpG islands. Genes are shown as boxes 144 above the axis for genes on the positive strand and below the axis for genes on the negative strands. Protein binding site clusters are shown as boxes 146 wherein the height of each box indicates the log likelihood score for the particular cluster. CpG islands are shown as boxes 148 in FIG. 3. For simplicity, only selected genes, protein binding site clusters, CpG islands are referenced in FIG. 3.

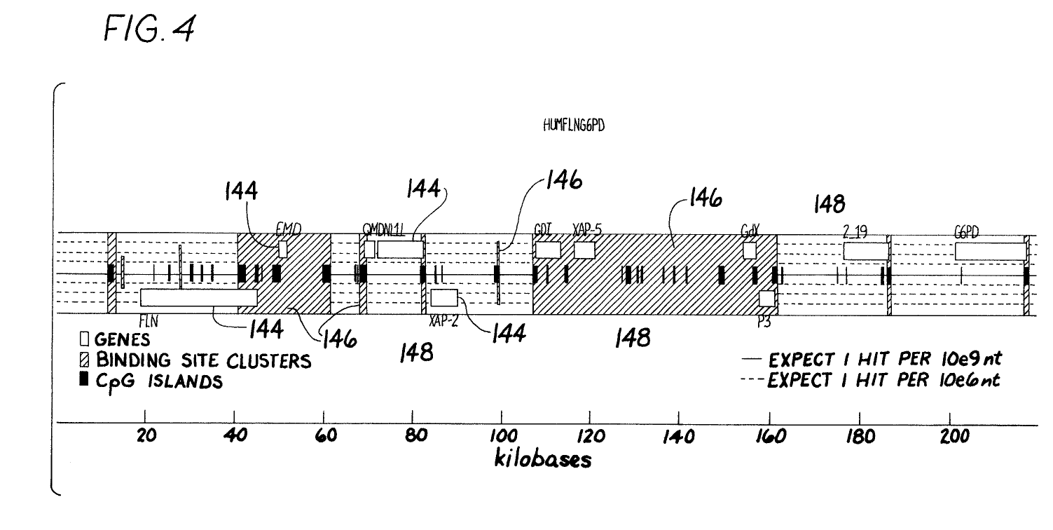

Similarly, FIG. 4 shows an analysis of the G6PD region on human Xq28 (GenBank accession HUMFLNG6PD). In this figure, the locations of annotated genes, protein binding site clusters, and CpG islands are also shown. Genes are shown again as boxes 144 above the axis for genes on the positive strand and below the axis for genes on the negative strands. Likewise, protein binding site clusters are shown as boxes 146 and CpG islands are shown as boxes 148. FIG. 4 references only some of the genes, binding site clusters, and CpG islands shown for simplicity.

To assess the functional correlates of the transcription factor cluster identified in scanning genomic sequence, the well annotated G6PD region of chromosome Xq28 was examined. This is a region with a high G+C content and high gene density. By both G+C content and gene density, it is in an H3 isochore. Twelve clusters of transcription factor binding sites with scores greater than 16 bits were identified in this region. All transcription factor clusters are within 10 kilobases of an annotated transcription start site, and 5 are within 1.5 kilobases. Not all genes are closely associated with an identified transcription factor binding site clusters. Similarly, in the human class 1 MHC locus, 12 protein-binding site clusters scoring above 16 bits were identified. Again, all clusters fall within 10 kilobases of a transcription start site and nine fall within 1.5 kilobases of a transcription start site.

### EXAMPLE 2

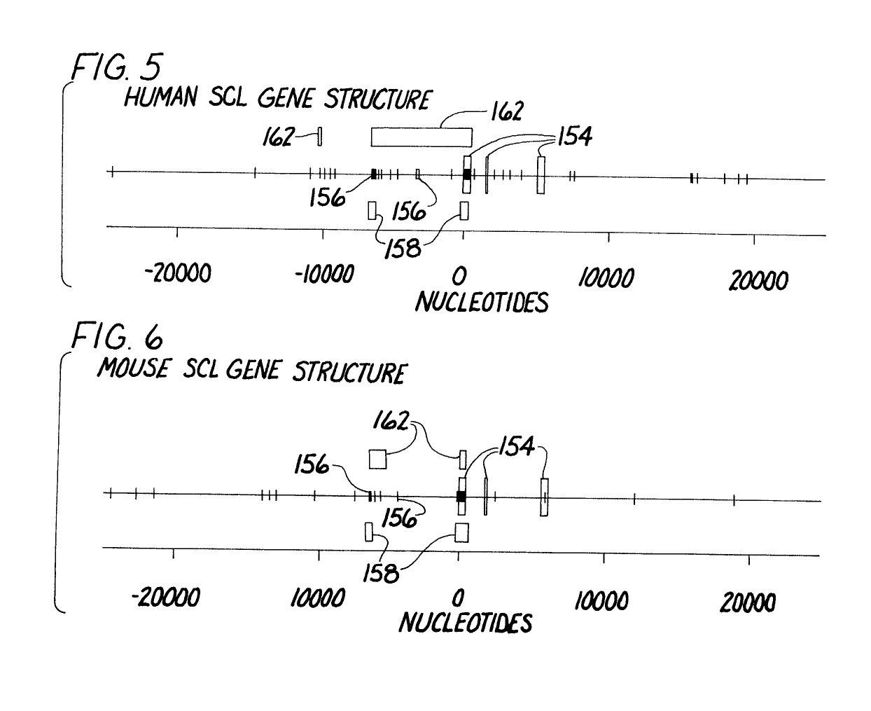

An evolutionarily conserved enhancer has been identified controlling the expression of the stem cell leukemia (SCL) gene, a transcription factor active in early hematopoesis. As is shown in FIGS. 5 and 6, high scoring clusters of protein binding sites are found in both the human and mouse SCL genes. Two clusters of binding sites are observed, one within the first coding exon, and the other approximately 7 kilobases upstream, at the transcription start site. The SCL gene has been subject to extensive experimental analysis. DNaseI hypersensitive sites are seen at sites corresponding to both transcription factor binding site clusters. Furthermore, both regions are conserved between chicken, mouse, and human. Interestingly, the transcription factors in the binding site cluster within exon I are similar to the binding sites in the upstream cluster. GATA-1 has been identified experimentally as an essential regulatory factor governing tissue specific expression of SCL. In this instance, GATA-1 site is in the high scoring binding site set at the transcription start site.

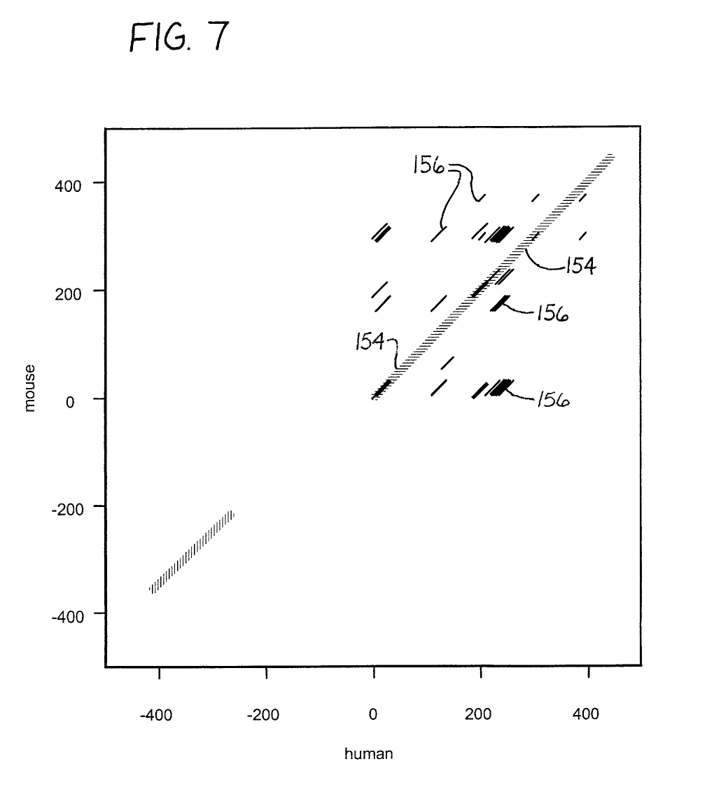

This example highlights the ambiguous nature of cluster boundaries; while two clusters are defined in the human gene, they are merged into a single cluster in the mouse gene. Second, detailed analysis of the binding sites (see FIG. 7) shows that considerable variation in precise binding site location may occur even when the proteins bound to a binding site are conserved.

FIGS. 5 and 6 provide a comparison of the analysis of human and mouse SCL genes, respectively. In both cases, a 50 kilobase window is shown with the start of transcription located at the origin. The three coding exons in this example are shown as boxes 154 on the axis. Individual protein binding sites scoring above 16 bits are shown as vertical bars 156 across the axis. Two CpG islands are shown as boxes 158 below the axis, one near the start of transcription and the other approximately 7 kilobases upstream. Protein binding site clusters are shown as boxes 162 above the axis. Both CpG islands are associated with statistically significant protein binding site clusters. In the case of the mouse sequence of FIG. 6, the two regions are joined into a single cluster.

Referring now to FIG. 7, a two-dimensional comparison of the gene structure is shown for 1 kilobase around the transcription start site. In FIG. 7, exons are again shown at reference character 154. Conserved sequence segments identified by BLASTN (expect<0.01) are shown at reference character 164 (a BLASTN search compares a nucleotide query sequence against a nucleotide sequence database). Pairs of sites scoring above 16 bits in both human and mouse are shown as black lines 156.

In this example, alignment of the first exonic region has no gaps and 88% identity at a nucleotide level (94% identity of the amino acid sequence). If the protein binding sites were strictly homologous, the matched sites would all fall precisely on the diagonal. Instead, many sites binding the same protein are seen off the diagonal indicating gain or loss of a site during the evolutionary processes separating humans and mice. Thus, while many of the factors bound in this cluster have been preserved, the precise order of the sites has not been. This would be consistent with convergent evolution of new sites.

Although described in connection with an exemplary computing system environment, the invention is operational with numerous other general purpose or special purpose computing system environments or configurations. The computing system environment is not intended to suggest any limitation as to the scope of use or functionality of the invention. Moreover, the computing system environment should not be interpreted as having any dependency or requirement relating to any one or combination of components illustrated in the exemplary operating environment. Examples of well known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The invention may be described in the general context of computer-executable instructions, such as program modules, executed by one or more computers or other devices. Generally, program modules include, but are not limited to, routines, programs, objects, components, and data structures that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained.

As various changes could be made in the above constructions and methods without departing from the scope of the invention, it is intended that all matter contained in the above description and shown in the accompanying drawings shall be interpreted as illustrative and not in a limiting sense.

What is claimed is:

1. A method of identifying clusters of protein binding sites in a nucleotide sequence under analysis, each protein binding site having a sequence that corresponds to a portion of the nucleotide sequence under analysis, said method comprising:
determining likelihood parameters for a plurality of known protein binding sites, said likelihood parameter for each protein binding site representing a likelihood that the protein binding site will occur in the nucleotide sequence under analysis relative to a likelihood that the protein binding site will occur in a random nucleotide sequence of a substantially equivalent composition;
grouping selected protein binding sites as a function of their respective likelihood parameters to determine a likelihood score;
comparing the likelihood score to a predetermined threshold; and
identifying the selected protein binding sites in the nucleotide sequence as one or more clusters if the likelihood score exceeds the predetermined threshold.

2. The method of claim 1 further comprising comparing the known protein binding sites to the nucleotide sequence under analysis to identify occurrences of one or more of the protein binding sites in the sequence.

3. The method of claim 2 further comprising generating a random nucleotide sequence and comparing the known protein binding sites to the random nucleotide sequence to identify occurrences of one or more of the protein binding sites in the random sequence.

4. The method of claim 3 wherein the likelihood score is a function of the respective occurrences of protein binding sites in the nucleotide sequence under analysis and the random nucleotide sequence.

5. The method of claim 1 wherein the known protein binding sites have a relative entropy of at least 8 bits.

6. The method of claim 1 wherein the threshold represents a level at which random occurrences of the known protein binding sites in the nucleotide sequence under analysis are highly unlikely.

7. The method of claim 1 wherein grouping the selected protein binding sites includes defining one or more groups of the selected protein binding sites wherein the protein binding sites are non-overlapping and selecting one or more sets of the selected protein binding sites to optimize the likelihood score.

8. The method of claim 7 wherein defining the one or more groups includes grouping non-overlapping protein binding sites according to a waiting time distribution.

9. The method of claim 8 wherein the waiting time distribution is defined by the following:
$$g ( t ) = \frac{{α}_{1}}{{l}_{1}} exp ( - \frac{t}{{l}_{1}} ) + \frac{{α}_{2}}{{l}_{2}} exp ( - \frac{t}{{l}_{2}} ) .$$

10. The method of claim 1 further comprising the step of annotating genes as a function of the identified clusters of protein binding sites.

11. The method of claim 1 wherein the nucleotide sequence under analysis has an expected dinucleotide frequency and wherein determining the likelihood parameters includes generating a null model as a function of the dinucleotide frequency of the nucleotide sequence, said null model representing a likelihood of that the protein binding site will randomly occur in the nucleotide sequence.

12. The method of claim 1 wherein determining the likelihood parameters includes deriving a first order Markov for the nucleotide sequence to represent the likelihood that the protein binding site will randomly occur in the nucleotide sequence.

13. The method of claim 1 wherein determining the likelihood parameters comprises determining a log likelihood, L(S), according to the following:
$$L ( S ) = {\sum }_{i = 1 , l} log ( {p}_{i} ( {s}_{i} ) ) - log ( f ( {s}_{1} ) ) {\sum }_{i = 2 , l} log ( f ( {s}_{i} \vert {s}_{i - 1} ) )$$
where L(S) is the log likelihood score for matching the nucleotide sequence S composed of residues sᵢ to sₘ with the protein binding site sequence of length l; sᵢ is a residue at position i in the nucleotide sequence; pᵢ(s) is the probability of the residue s at position i in the protein binding site sequence; f(s) is the frequency of the residue s in the nucleotide sequence as a whole; and f (s|s′) is the conditional probability for finding the residue s in the nucleotide sequence as a whole given previous residues s′.

14. The method of claim 1 further comprising defining an index that references segments of the nucleotide sequence and searching the index to find the segments that are similar to one or more of the selected protein binding sites.

15. The method of claim 14 wherein the index has a plurality of locations, each location of the index referencing a plurality of the segments of the nucleotide sequence.

16. The method of claim 14 wherein searching the index includes defining a query sequence and identifying homologs to the query sequence.

17. The method of claim 1 further comprising identifying disease associations in the identified clusters.

18. A computer readable medium having computer-executable instructions for performing the method of claim 1.

19. A method of identifying protein binding sites in a nucleotide sequence under analysis, each identified protein binding site having a sequence that corresponds to a portion of the nucleotide sequence under analysis, said method comprising the steps of:
determining likelihood parameters for a plurality of known protein binding sites, said likelihood parameter for each protein binding site representing a likelihood that the protein binding site will occur in the nucleotide sequence binding site will occur in a random nucleotide sequence of a substantially equivalent composition;
comparing the likelihood parameters to a predetermined threshold to select the protein binding sites that have a substantially greater relative likelihood of occurrence;
defining an index that references segments of the nucleotide sequence;
searching the index to find the segments that are similar to one or more of the selected protein binding sites; and
identifying the segments found in the index search as protein binding sites in the nucleotide sequence based on the index search.

20. The method of claim 19 further comprising the steps of:
defining one or more sets of the selected protein binding sites wherein the protein binding sites are non-overlapping;
determining a cumulative likelihood parameter for each set of the selected protein binding sites;
selecting one or more sets of the selected protein binding sites to optimize the cumulative likelihood parameter; and
defining the selected sets of the selected protein binding sites to be clusters.

21. The method of claim 19 wherein the nucleotide sequence under analysis has an expected dinucleotide frequency and wherein the step of determining the likelihood parameter includes generating a null model as a function of the dinucleotide frequency of the nucleotide sequence, said null model representing a likelihood of that the protein binding site will randomly occur in the nucleotide sequence.

22. A computer readable medium having stored thereon a data structure, said data structure for use in reporting protein binding site clusters, said data structure comprising:
a first field containing individual protein binding sites information, said individual protein binding sites being identified in a nucleotide sequence under analysis, each protein binding site having a sequence that corresponds to a portion of the nucleotide sequence under analysis;
a second field containing cluster information identifying clusters of the protein binding sites in the nucleotide sequence under analysis, said clusters being identified from the protein binding sites as a function of likelihood parameters for the protein binding sites, said likelihood parameter for each protein binding site representing a likelihood that the protein binding site will occur in the nucleotide sequence under analysis relative to a likelihood that the protein binding site will occur in a random nucleotide sequence of a substantially equivalent composition.

23. The data structure of claim 22 further comprising:
a third field containing grouped protein binding sites information, said protein binding sites being grouped as a function of their respective likelihood parameters.

24. The data structure of claim 23 further comprising:
a fourth field containing likelihood score for the grouped protein binding sites, said clusters in the second field being identified from the protein binding sites as one or more clusters if their respective likelihood scores exceed a predetermined threshold.
